(19) United States
(12) Patent Application Publication
(10) Pub. No.: US 2021/0061870 A1
(43) Pub. Date: Mar. 4, 2021

(54) METHOD AND SYSTEM FOR EXTRACTING NEOANTIGENS FOR IMMUNOTHERAPY

(71) Applicant: Shenzhen NeoCura Biotechnology Corporation, Shenzhen (CN)

(72) Inventors: Ji WAN, Shenzhen (CN); Qi SONG, Shenzhen (CN); Youdong PAN, Shenzhen (CN); Di XIA, Shenzhen (CN); Peng LIU, Shenzhen (CN); Jian WANG, Shenzhen (CN)

(73) Assignee: Shenzhen NeoCura Biotechnology Corporation, Shenzhen (CN)

(21) Appl. No.: 16/991,042

(22) Filed: Aug. 12, 2020

(30) Foreign Application Priority Data
Sep. 2, 2019 (CN) 201910823630.1

(51) Int. Cl. C07K 14/47; A61K 39/00

(52) U.S. Cl. CPC C07K 14/4748; C40B 40/10; A61K 39/0011

(57) ABSTRACT

A method and system for extracting neoantigens for immunotherapy includes the following steps: step S1: acquiring conventional proteomes of tumor tissue and normal tissue samples; step S2: acquiring nucleotide polymer sequence libraries of the tumor tissue and normal tissue samples and a specific proteome of the tumor tissue sample; step S3: acquiring a plurality of candidate tumor-specific neoantigens based on the conventional and specific proteomes of the tumor tissue sample and molecular human leukocyte antigen (HLA) typing; and step S4: calculating the presence of the plurality of candidate tumor-specific neoantigens in the conventional proteomes and the nucleotide polymer sequence libraries of the tumor tissue and normal tissue samples, and acquiring tumor-specific neoantigens with a multiple of gene expression changes as a filter rule. More tumor-specific neoantigens are discovered using the new method because they are not limited to coding regions and are partly derived from genome noncoding regions (NCRs).

### CROSS REFERENCES TO THE RELATED APPLICATIONS

This application is based upon and claims priority to Chinese Patent Application No. 201910823630.1, filed on Sep. 2, 2019, the entire contents of which are incorporated herein by reference.

### TECHNICAL FIELD

The present invention relates to the technical field of tumor immunotherapy, and in particular, to a method and system for extracting neoantigens for immunotherapy.

### BACKGROUND

At present, malignancy is one of the diseases most seriously harmful to human beings. Therapies for malignancies have been constantly improving and developing over the past few decades. So far, conventional therapies for malignancies include surgery, radiotherapy, chemotherapy, and targeted therapy. Current therapeutic regimens, however, have limitations, such as toxicity and other harmful side effects, including tumor recurrence.

Most recently, immunotherapies that activate the immune system to inhibit and kill tumor cells have become especially promising in the field of malignancy. Principal immunotherapies can be classified into three classes according to the mechanisms thereof:

(1) immune checkpoint inhibitors, which retard inhibitory signals of the immune system to activate the immune system;

(2) adoptive cellular immunotherapy (ACI), which modifies T lymphocytes to recognize specific antigens; and

(3) neoantigen-based immunotherapies, which predict tumor-specific antigens, so that a vaccine may be prepared or T cells propagated in vitro and reintroduced into the body according to the specific antigen predicted.

Compared with immune checkpoint inhibitors and ACT, neoantigen-based immunotherapies are more widely applicable, less toxic and have fewer side effects. Thus far, prediction of neoantigen-based therapies typically includes: analysis of data for whole exome sequencing (WES) and transcriptome resequencing of tumor and normal tissues; identification of DNA mutations in protein-coding regions and subtypes of human leucocyte antigen (HLA); acquisition of mutated polypeptides translated from mutated DNAs by bioinformatics method; and final prediction of whether the mutated polypeptides can be presented to the cell surface by HLA. Neoantigens predicted by the above methods exhibit excellent clinical effects on tumors (i.e., melanoma) with larger tumor mutation burden (TMB). With respect to malignant tumors with lower TMB, however, the selection of tumor neoantigen vaccine formulations is limited due to the small number of predicted neoantigens. Therefore, it is highly desirable to expand the screening range of the existing neoantigen prediction, which has important implications in the efficacy of neoantigens.

### SUMMARY

In view of the above-mentioned problem and in consideration of the possibility that tumor-specific RNAs annotated as nonprotein coding regions produce mutated polypeptides, the present invention provides a method for extracting neoantigens for immunotherapy.

The present invention provides a method for extracting neoantigens for immunotherapy, including:

step S1: acquiring conventional proteomes of tumor tissue and normal tissue samples;

step S2: acquiring nucleotide polymer sequence libraries of the tumor tissue and normal tissue samples and a specific proteome of the tumor tissue sample;

step S3: acquiring a plurality of candidate tumor-specific neoantigens based on the conventional proteome and the specific proteome of the tumor tissue sample, and molecular human leukocyte antigen (HLA) typing; and

step S4: separately calculating feature values of the plurality of candidate tumor-specific neoantigens based on the plurality of candidate tumor-specific neoantigens acquired, and acquiring tumor-specific neoantigens by filtering under a preset rule.

Optionally, the step S1 of acquiring conventional proteomes of tumor tissue and normal tissue samples includes:

step S11: detecting point mutations of transcripts of the tumor tissue and normal tissue samples;

step S12: calculating expression levels of transcripts in the tumor tissue and normal tissue samples;

step S13: constructing mutated exomes of the tumor tissue and normal tissue samples; and

step S14: translating the mutated exomes of the tumor tissue and normal tissue samples.

Optionally, the step S2 of acquiring nucleotide polymer sequence libraries of the tumor tissue and normal tissue samples and a specific proteome of the tumor tissue sample includes:

step S21: generating nucleotide polymer sequence libraries of preset length;

step S22: acquiring tumor-specific nucleotide polymer sequences;

step S23: assembling the tumor-specific nucleotide polymer sequences; and

step S24: conducting reading frame translation on assembled tumor-specific sequences.

Optionally, the step S3 of acquiring a plurality of candidate tumor-specific neoantigens based on the conventional proteome and the specific proteome of the tumor tissue sample, and molecular HLA typing includes:

step S31: acquiring the molecular HLA typing;

step S32: generating a global tumor proteome based on the determined conventional and specific proteomes of the tumor tissue sample;

step S33: predicting HLA-peptide binding affinity scores to acquire a target peptide sequence using the acquired global tumor proteome and the molecular HLA typing result; and

step S34: annotating characteristics of the target peptide sequence to acquire candidate tumor-specific neoantigens.

The present invention further provides a system for extracting neoantigens for immunotherapy, including:

a conventional proteome acquiring unit, used for acquiring conventional proteomes of tumor tissue and normal tissue samples;

a specific proteome acquiring unit, used for acquiring nucleotide polymer sequence libraries of the tumor tissue and normal tissue samples and a specific proteome of the tumor tissue sample;

a candidate neoantigen determining unit, used for acquiring a plurality of candidate tumor-specific neoantigens based on the conventional proteome and the specific proteome of the tumor tissue sample, and molecular human leukocyte antigen (HLA) typing; and

a tumor-specific neoantigen determining unit, used for separately calculating feature values of the candidate tumor-specific neoantigens based on the plurality of candidate tumor-specific neoantigens acquired, and acquisition of tumor-specific neoantigens by filtering under a preset rule.

Optionally, the conventional proteome acquiring unit includes:

a detection subunit, used for detecting point mutations of transcripts of the tumor tissue and normal tissue samples;

a calculation subunit, used for calculating expression levels of transcripts in the tumor tissue and normal tissue samples;

a construction subunit, used for constructing mutated exomes of the tumor tissue and normal tissue samples; and

a translation subunit, used for translating the mutated exomes of the tumor tissue and normal tissue samples.

Optionally, the specific proteome acquiring unit includes:

a generation subunit, used for generating nucleotide polymer sequence libraries of preset length;

an acquisition subunit, used for acquiring tumor-specific nucleotide polymer sequences;

an assembly subunit, used for assembling the tumor-specific nucleotide polymer sequences; and

a reading frame translation subunit, used for reading frame translation of tumor-specific sequences.

Optionally, the candidate neoantigen determining unit includes:

an HLA acquiring subunit, used for acquiring the molecular HLA typing;

a global tumor proteome generating subunit, used for generating a global tumor proteome based on the determined conventional and specific proteomes of the tumor tissue sample;

a target peptide sequence acquiring subunit, used for predicting HLA-peptide binding affinity scores to acquire a target peptide sequence using the acquired global tumor proteome and the molecular HLA typing result; and

a candidate tumor-specific neoantigen acquiring subunit, used for annotating characteristics of the target peptide sequence to acquire candidate tumor-specific neoantigens.

Compared with the prior art, the present invention has the following advantages:

I. With respect to their source, tumor-specific neoantigens discovered using the new method of the invention are not limited to coding regions and partly derived from noncoding genomics regions (NCRs). More neoantigens are discovered as a result. At present, methods typically include target sequencing and whole exome sequencing, by which neoantigens are acquired by affinity prediction after recognition of somatic variation; in this way, regions to be analyzed are limited to only coding regions in a genome.

II. The majority of tumor-specific neoantigens acquired by the present method are derived from non-mutated, highly expressed transcripts (e.g., endogenous reverse transcription). As a result, tumor-specific neoantigens are universal in different tumor types.

Other features and advantages of the disclosure will be described in the following description, and some of these will become apparent from the description or be understood by implementing the invention. The objectives and other advantages of the invention can be implemented or obtained by structures specifically indicated in the written description, claims, and accompanying drawings.

The technical solution of the present invention is further described in detail below with reference to the accompanying drawings and examples.

### BRIEF DESCRIPTION OF THE DRAWINGS

The accompanying drawings are used to provide further understanding of the present invention and constitute a part of the specification. The accompanying drawings, together with the examples of the present invention, are used to explain the present invention but do not pose a limitation to the present invention. In the accompanying drawings:

FIG. 1 is a schematic diagram of a method for extracting neoantigens for immunotherapy in an example of the present invention;

FIG. 2 is a schematic diagram of acquisition of conventional proteomes of tumor tissue and normal tissue samples in an example of the present invention;

FIG. 3 is a schematic diagram of acquisition of nucleotide polymer sequence libraries of tumor tissue and normal tissue samples and a specific proteome of the tumor tissue sample in an example of the present invention;

FIG. 4 is a schematic diagram of acquisition of candidate tumor-specific neoantigens in an example of the present invention; and

FIG. 5 is a schematic diagram of a system for extracting neoantigens for immunotherapy in an example of the present invention.

### REFERENCE NUMERALS

41. Conventional proteome acquiring unit; 42. specific proteome acquiring unit; 43. candidate neoantigen determining unit; and 44. tumor-specific neoantigen determining unit.

### DETAILED DESCRIPTION OF THE EMBODIMENTS

The preferred examples of the present invention are described below with reference to the accompanying drawings. It should be understood that the preferred examples described herein are only used to illustrate and explain the present invention and are not intended to limit the present invention.

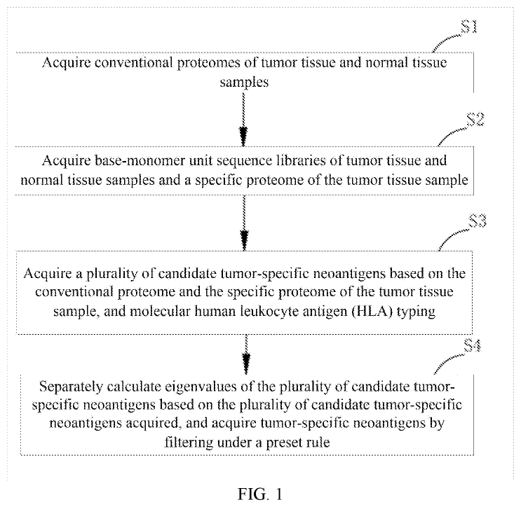

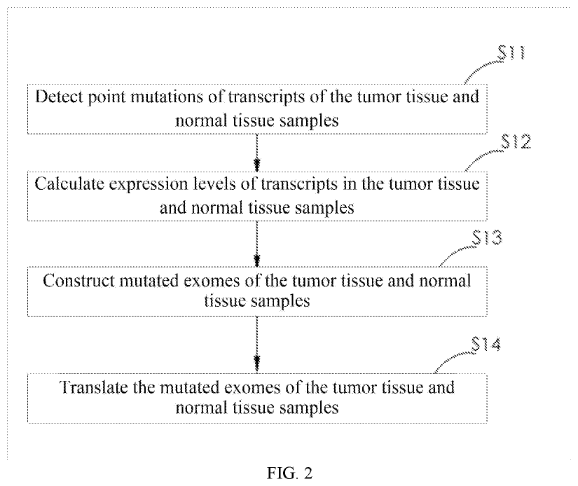

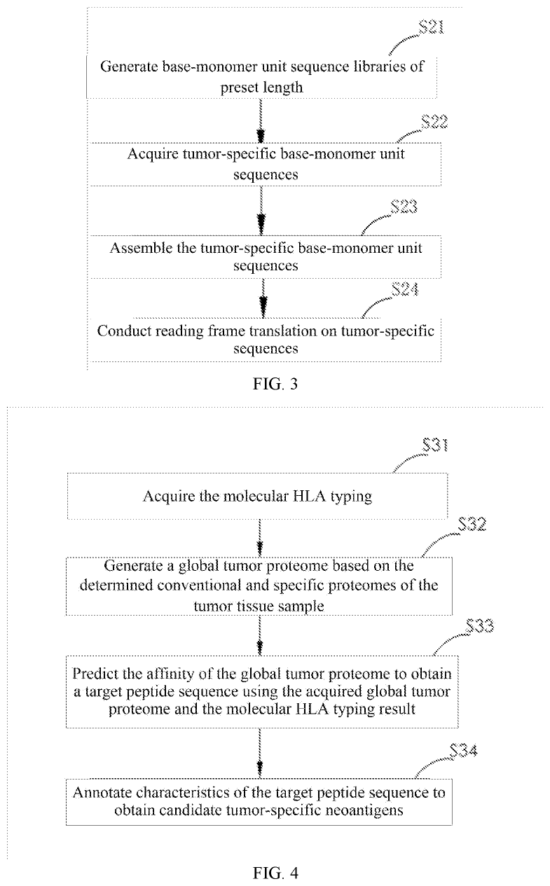

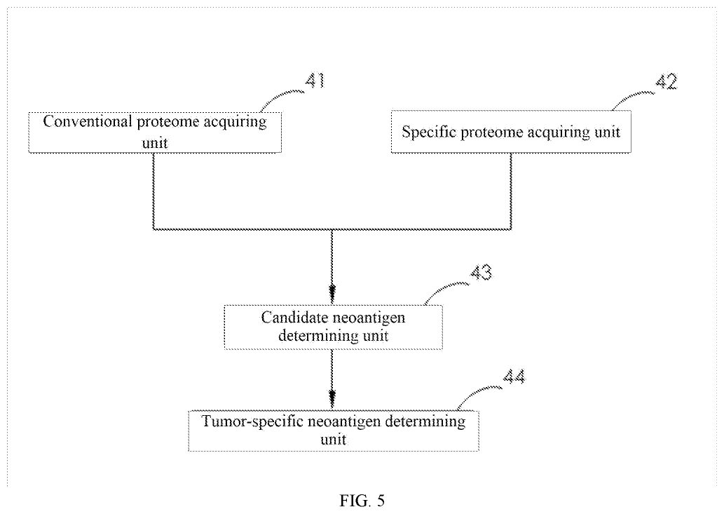

A schematic diagram of a method for extracting neoantigens for immunotherapy is provided in an example of the present invention. As shown in FIG. 1, the method includes the following steps.

Step S1: acquire conventional proteomes of tumor tissue and normal tissue samples.

Step S2: acquire nucleotide polymer sequence libraries of the tumor tissue and normal tissue samples and a specific proteome of the tumor tissue sample.

Step S3: acquire a plurality of candidate tumor-specific neoantigens based on the conventional proteome and the specific proteome of the tumor tissue sample, and molecular human leukocyte antigen (HLA) typing.

Step S4: separately calculate feature values of the plurality of candidate tumor-specific neoantigens based on the plurality of candidate tumor-specific neoantigens acquired, and acquire tumor-specific neoantigens with a multiple of gene expression changes as a filter rule.

The operating principle and beneficial effects of the above technical solution are as follows:

Candidate tumor-specific neoantigens are acquired based on the conventional proteome and the specific proteome of the tumor tissue sample and molecular HLA typing. Subsequently, feature values of the plurality of candidate tumor-specific neoantigens are separately calculated based on the candidate tumor-specific neoantigens acquired. Feature values represent the presence of candidate tumor-specific neoantigens in the conventional proteomes of the tumor tissue and normal tissue samples, and the nucleotide polymer sequence libraries of the tumor tissue and normal tissue samples. If present, the feature value is expressed as 1; if absent, the feature value is expressed as 0. The four feature values are combined into a feature vector for judgment, and tumor-specific neoantigens are acquired with a multiple (20-fold) of gene expression changes as a filter rule. Thus, this realizes the discovery of tumor-specific neoantigens in genome noncoding regions (NCRs).

With regard to their source, the tumor-specific neoantigens discovered by the methods of the invention are not limited to coding regions and partly derived from genome NCRs. Therefore, more neoantigens are discovered. At present, common methods principally include target sequencing and whole exome sequencing, by which neoantigens are acquired by affinity prediction after recognition of somatic variation; in this way, regions to be analyzed are limited to coding regions in a genome.

The majority of tumor-specific neoantigens acquired are derived from non-mutated, highly expressed transcripts (e.g., endogenous reverse transcription). Therefore, tumor-specific neoantigens are universal in different tumor types.

In an example, the step S1 of acquiring conventional proteomes of tumor tissue and normal tissue samples includes the following steps.

Step S11: detect point mutations of transcripts of the tumor tissue and normal tissue samples.

First, raw high-throughput next-generation sequencing (NGS) data filtering is essential for subsequent analysis, which removes some useless sequences to improve the accuracy and efficiency of the subsequent analysis. Specifically, the raw data are filtered by using sequencing data filtering software Trimmomatic.

Next, the filtered data are mapped into a reference genome using sequence alignment software Star; subsequently, mutation is identified by mutation recognition program Freebayes.

Step S12: calculate expression levels of transcripts in the tumor tissue and normal tissue samples.

Specifically, each transcript is expressed quantitatively by using sequence quantification software Kallisto.

Step S13: construct mutated exomes of the tumor tissue and normal tissue samples.

Specifically, using program package Pygeno, mutations with a base quality of >20 in variant calling results are constructed into mutated exomes of tumor tissue and normal tissue samples, respectively.

Step S14: translate the mutated exomes of the tumor tissue and normal tissue samples.

First, transcripts with expression greater than 0 are selected according to results of expression analysis of transcripts and are translated into protein sequences of the tumor tissue and normal tissue samples using the constructed mutated exomes of the tumor tissue and normal tissue samples.

Next, to enable the results to be used in the analysis process of acquiring the specific proteome of the tumor tissue sample, translation results need to be reformatted.

In an example, the step S2 of acquiring nucleotide polymer sequence libraries of the tumor tissue and normal tissue samples and a specific proteome of the tumor tissue sample includes the following steps.

Step S21: generate nucleotide polymer sequence libraries of preset length.

According to the sequencing data of the samples, Jellyfish software is used to acquire nucleotide polymer sequence libraries of the tumor tissue and normal tissue samples three times as long as the theoretical length range (8-12 amino acids) of class I HLA epitope peptides, where selection of the length of nucleotide polymer unit should be noted.

Step S22: acquire tumor-specific nucleotide polymer sequences.

A specific nucleotide polymer sequence in the tumor tissue sample is selected according to the presence of nucleotide polymer sequences in the tumor tissue and normal tissue samples.

Step S23: assemble the tumor-specific nucleotide polymer sequences.

Tumor-specific nucleotide polymer unit is assembled to acquire tumor-specific sequences using de novo assembly software Nektar assembly.

Step S24: conduct reading frame translation on assembled tumor-specific sequences.

Reading frame translation is conducted on the above assembled tumor-specific sequences to acquire tumor-specific amino acid sequences. The present invention selects sequences with a length of more than 8 amino acids.

In an example, the step S3 of acquiring a plurality of candidate tumor-specific neoantigens based on the conventional proteome and the specific proteome of the tumor tissue sample, and molecular human leukocyte antigen (HLA) typing includes the following steps.

Step S31: acquire the molecular HLA typing.

Molecular HLA typing is calculated by molecular HLA typing software HLA-LA.

Step S32: generate a global tumor proteome based on the determined conventional and specific proteomes of the tumor tissue sample.

Conventional and specific proteomes of the tumor tissue sample are combined. The data generated thereby are named the global tumor proteome.

Step S33: predict HLA-peptide binding affinity scores to acquire a target peptide sequence using the acquired global tumor proteome and the molecular HLA typing result.

The HLA-peptide binding affinity scores are predicted to acquire a target peptide sequence using NetMHCPan-4.0 software and the molecular HLA typing result.

Step S34: annotate characteristics of the target peptide sequence to acquire candidate tumor-specific neoantigens. The target peptide is annotated as a characteristic of the candidate tumor-specific neoantigen.

In the present invention, feature values of the plurality of candidate tumor-specific neoantigens are calculated separately, and tumor-specific neoantigens are acquired by filtering under a preset rule. Details include:

To acquire candidate tumor-specific neoantigens, coding sequences of all peptide fragments are queried from the conventional proteomes of the tumor tissue and normal tissue samples and the nucleotide polymer sequence libraries of the tumor tissue and normal tissue samples, respectively. If present in the database, the result is expressed as 1; if absent, the result is expressed as 0. The four feature values are combined into a feature vector for judgment. In the present invention, regardless of detection status, coding sequences thereof are excluded from peptide fragments detected in the conventional proteome of the normal tissue sample. This is because these coding sequences lead to tolerance, i.e., if a feature vector is [*, 1, *, *] (* is 0 or 1), all coding sequences are excluded. Truly tumor-specific peptide fragments should not be detected in the normal tissue sample. In other words, these peptide fragments are not detected in either conventional proteome of the normal tissue sample or the nucleotide polymer sequence library of the normal tissue sample. That is, if the corresponding feature vectors are [1, 0, 1, 0] and [0, 0, 1, 0], candidate tumor-specific neoantigens with these peptide fragments can be labeled as tumor-specific neoantigens. Truly tumor-specific peptide fragments should not be detected in the normal tissue sample. In other words, these peptide fragments are not detected in either conventional proteome of the normal tissue sample or the nucleotide polymer sequence library of the normal tissue sample. That is, if the corresponding feature vectors are [1, 0, 1, 1], candidate tumor-specific neoantigens with these peptide fragments can be labeled as tumor-specific neoantigens. If peptide fragments are absent in the conventional proteomes of the normal tissue and tumor tissue samples, but present in the nucleotide polymer sequence libraries of the normal tissue and tumor tissue samples, the corresponding feature vectors are [0, 0, 1, 1]; RNA coding sequences cannot be labeled until expression of these sequences in tumor cells are at least 20-fold higher than that in normal cells. Finally, coding sequences of peptide fragments of RNA sequences derived from different proteins are consistent, which can further be labeled as candidate tumor-specific neoantigens.

The present invention further provides a system for extracting neoantigens for immunotherapy, including:

a conventional proteome acquiring unit 41 for acquiring conventional proteomes of tumor tissue and normal tissue samples;

a specific proteome acquiring unit 42 for acquiring nucleotide polymer sequence libraries of the tumor tissue and normal tissue samples and a specific proteome of the tumor tissue sample;

a candidate neoantigen determining unit 43 for acquiring a plurality of candidate tumor-specific neoantigens based on the conventional and specific proteome of the tumor tissue sample and molecular HLA typing; and

a tumor-specific neoantigen determining unit 44, used for separately calculating feature values of the candidate tumor-specific neoantigens based on the plurality of candidate tumor-specific neoantigens acquired, and acquisition of tumor-specific neoantigens by filtering under a preset rule. Thus, this realizes the discovery of tumor-specific neoantigens in genome NCRs.

The operating principle and beneficial effects of the above technical solution are as follows: first, the conventional proteome acquiring unit acquires the conventional proteomes of the tumor tissue and normal tissue samples, and the specific proteome acquiring unit acquires the nucleotide polymer sequence libraries of the tumor tissue and normal tissue samples and the specific proteome of the tumor tissue sample; next, the candidate neoantigen determining unit acquires candidate tumor-specific neoantigens based on the conventional proteome and the specific proteome of the tumor tissue sample, and molecular HLA typing; finally, feature values of the plurality of the candidate tumor-specific neoantigens are separately calculated based on the acquired candidate tumor-specific neoantigens. Feature values represent the presence of candidate tumor-specific neoantigens in the conventional proteomes of the tumor tissue and normal tissue samples, and the nucleotide polymer sequence libraries of the tumor tissue and normal tissue samples. If present, the feature value is expressed as 1; if absent, the feature value is expressed as 0. The four feature values are combined into a feature vector for judgment, and tumor-specific neoantigens are acquired with a multiple of gene expression changes as a filter rule. In view of source, the tumor-specific neoantigens discovered by the solution of the present invention are not limited to coding regions and partly derived from genome NCRs. Therefore, more neoantigens will be discovered. At present, common methods principally include target sequencing and whole exome sequencing, by which neoantigens are acquired by affinity prediction after recognition of somatic variation. In this way, regions to be analyzed are limited to coding regions in a genome.

The majority of tumor-specific neoantigens acquired are derived from non-mutated, highly expressed transcripts (e.g., endogenous reverse transcription). Therefore, tumor-specific neoantigens are universal in different tumor types.

In an example, the conventional proteome acquiring unit includes a detection subunit, a calculation subunit, a construction subunit, and a translation subunit.

The detection subunit is used for detecting point mutations of transcripts of the tumor tissue and normal tissue samples;

First, raw high-throughput NGS data filtering is essential for subsequent analysis, which removes some useless sequences to improve the accuracy and efficiency of the subsequent analysis. Specifically, the raw data are filtered by using sequencing data filtering software Trimmomatic.

Next, the filtered data are mapped into a reference genome using sequence alignment software Star; subsequently, mutation is identified by mutation recognition program Freebayes.

The calculation subunit is used for calculating expression levels of transcripts in the tumor tissue and normal tissue samples;

Specifically, each transcript is expressed quantitatively by using sequence quantification software Kallisto.

The construction subunit is used for constructing mutated exomes of the tumor tissue and normal tissue samples.

Specifically, using program package Pygeno, mutations with a base quality of >20 in variant calling results are constructed into mutated exomes of tumor tissue and normal tissue samples, respectively.

The translation subunit is used for translating the mutated exomes of the tumor tissue and normal tissue samples.

First, transcripts with expression greater than 0 are selected according to results of expression analysis of transcripts and are translated into protein sequences of the tumor tissue and normal tissue samples using the constructed mutated exomes of the tumor tissue and normal tissue samples.

Next, to enable the results to be used in the analysis process of acquiring the specific proteome of the tumor tissue sample, translation results need to be reformatted.

In an example, the specific proteome acquiring unit includes a generation subunit, an acquisition subunit, an assembly subunit, and a reading frame translation subunit.

The generation subunit is used for generating nucleotide polymer sequence libraries of preset length. According to the sequencing data of the samples, Jellyfish software is used to acquire nucleotide polymer sequence libraries of the tumor tissue and normal tissue samples three times as long as the theoretical length range (8-12 amino acids) of class I HLA epitope peptides, where selection of the length of nucleotide polymer unit should be noted.

The acquisition subunit is used for acquiring tumor-specific nucleotide polymer sequences.

A specific nucleotide polymer sequence in the tumor tissue sample is selected according to the presence of nucleotide polymer sequences in the tumor tissue and normal tissue samples.

The assembly subunit is used for assembling the tumor-specific nucleotide polymer sequences.

Tumor-specific nucleotide polymer units are assembled to acquire tumor-specific sequences using de novo assembly software Nektar assembly.

The reading frame translation subunit is used for reading frame translation of assembled tumor-specific sequences.

Reading frame translation is conducted on the above assembled tumor-specific sequences to acquire tumor-specific amino acid sequences. The present invention selects sequences with a length of more than 8 amino acids.

In an example, the candidate neoantigen determining unit includes an HLA acquiring subunit, a global tumor proteome generating subunit, a target peptide sequence acquiring subunit and a candidate tumor-specific neoantigen acquiring subunit.

The HLA acquiring subunit is used for acquiring the molecular HLA typing.

Molecular HLA typing is calculated by molecular HLA typing software HLA-LA.

The global tumor proteome generating subunit is used for generating a global tumor proteome based on the determined conventional and specific proteomes of the tumor tissue sample.

Conventional and specific proteomes of the tumor tissue sample are combined. The data generated thereby are named the global tumor proteome.

The target peptide sequence acquiring subunit is used for predicting HLA-peptide binding affinity scores to acquire a target peptide sequence using the acquired global tumor proteome and the molecular HLA typing result.

The HLA-peptide binding affinity scores are predicted to acquire a target peptide sequence using NetMHCPan-4.0 software and the molecular HLA typing result.

The candidate tumor-specific neoantigen acquiring subunit is used for annotating characteristics of the target peptide sequence to acquire candidate tumor-specific neoantigens. The target peptide is annotated as a characteristic of the candidate tumor-specific neoantigen.

In the present invention, feature values of the plurality of candidate tumor-specific neoantigens are calculated separately, and tumor-specific neoantigens are acquired by filtering under a preset rule. Details include:

To acquire candidate tumor-specific neoantigens, coding sequences of all peptide fragments are queried from the conventional proteomes of the tumor tissue and normal tissue samples, and the nucleotide polymer sequence libraries of the tumor tissue and normal tissue samples based on annotated target peptide fragments of the acquired candidate tumor-specific neoantigens, respectively. If the annotated target peptide fragments are present in the database, the result is expressed as 1; if absent, the result is expressed as 0. The four feature values are combined into a feature vector for judgment. In the present invention, regardless of detection status, coding sequences thereof are excluded from peptide fragments detected in the conventional proteome of the normal tissue sample. This is because these coding sequences lead to tolerance, i.e., if a feature vector is [*, 1, *, *] (* is 0 or 1), all coding sequences are excluded. Truly tumor-specific peptide fragments should not be detected in the normal tissue sample. In other words, these peptide fragments are not detected in either conventional proteome of the normal tissue sample or the nucleotide polymer sequence library of the normal tissue sample. That is, if the corresponding feature vectors are [1, 0, 1, 0] and [0, 0, 1, 0], candidate tumor-specific neoantigens with these peptide fragments can be labeled as tumor-specific neoantigens. Truly tumor-specific peptide fragments should not be detected in the normal tissue sample. In other words, these peptide fragments are not detected in either conventional proteome of the normal tissue sample or the nucleotide polymer sequence library of the normal tissue sample. That is, if the corresponding feature vector is [1, 0, 1, 1], candidate tumor-specific neoantigens with these peptide fragments can be labeled as tumor-specific neoantigens. If peptide fragments are absent in the conventional proteomes of the normal tissue and tumor tissue samples, but present in the nucleotide polymer sequence libraries of the normal tissue and tumor tissue samples, the corresponding feature vector is [0, 0, 1, 1]; RNA coding sequences cannot be labeled until expression of these sequences in tumor cells are at least 20-fold higher than that in normal cells. Finally, coding sequences of peptide fragments of RNA sequences derived from different proteins are consistent, which can further be labeled as tumor-specific neoantigens.

Persons skilled in the art should understand that the embodiments of the present invention may be provided as a method, a system, or a computer program product. Therefore, the present invention may use a form of hardware only embodiments, software only embodiments, or embodiments with a combination of software and hardware. Moreover, the present invention may use a form of a computer program product that is implemented on one or more computer-usable storage media (including but not limited to a disk memory, CD-ROM, an optical memory, and the like) that include computer-usable program code.

The present invention is described with reference to the flowcharts and/or block diagrams of the method, the device (system), and the computer program product according to the embodiments of this application. It should be understood that computer program instructions may be used to implement each process and/or each block in the flowcharts and/or the block diagrams and a combination of a process and/or a block in the flowcharts and/or the block diagrams. These computer program instructions may be provided for a general-purpose computer, a dedicated computer, an embedded processor, or a processor of any other programmable data processing device to generate a machine, so that the instructions executed by a computer or a processor of any other programmable data processing device generate an apparatus for implementing a specific function in one or more processes in the flowcharts and/or in one or more blocks in the block diagrams.

These computer program instructions may also be stored in a computer readable memory that can instruct the computer or any other programmable data processing device to work in a specific manner, so that the instructions stored in the computer readable memory generate an artifact that includes an instruction apparatus. The instruction apparatus implements a specific function in one or more processes in the flowcharts and/or in one or more blocks in the block diagrams.

These computer program instructions may also be loaded onto a computer or another programmable data processing device, so that a series of operations and steps are performed on the computer or the another programmable device, thereby generating computer-implemented processing. Therefore, the instructions executed on the computer or the other programmable device provides steps for implementing a specific function in one or more processes in the flowcharts and/or in one or more blocks in the block diagrams.

Finally, for the purposes of promoting an understanding of the principles of the invention, specific embodiments have been described. It should nevertheless be understood that the description is intended to be illustrative and not restrictive in character, and that no limitation of the scope of the invention is intended. Any alterations and further modifications in the described components, elements, processes or devices, and any further applications of the principles of the invention as described herein, are contemplated as would normally occur to one skilled in the art to which the invention pertains.

What is claimed is:

1. A method for extracting neoantigens for immunotherapy, comprising:
step S1: acquiring a conventional proteome of a tumor tissue sample and a conventional proteome of a normal tissue sample;
step S2: acquiring nucleotide polymer sequence libraries of the tumor tissue sample and the normal tissue sample, and a specific proteome of the tumor tissue sample;
step S3: acquiring a plurality of candidate tumor-specific neoantigens based on the conventional proteome of the tumor tissue sample and the specific proteome of the tumor tissue sample, and acquiring molecular human leukocyte antigen (HLA) typing; and
step S4: separately calculating feature values of the plurality of candidate tumor-specific neoantigens based on the plurality of candidate tumor-specific neoantigens acquired, and acquiring tumor-specific neoantigens by filtering under a preset rule.

2. The method for extracting neoantigens for immunotherapy according to claim 1, wherein the step S1 of acquiring the conventional proteome of the tumor tissue sample and the conventional proteome of the normal tissue sample comprises:
step S11: detecting point mutations of transcripts of the tumor tissue sample and the normal tissue sample;
step S12: calculating expression levels of transcripts in the tumor tissue sample and the normal tissue sample;
step S13: constructing mutated exomes of the tumor tissue sample and the normal tissue sample; and
step S14: translating the mutated exomes of the tumor tissue sample and the normal tissue sample.

3. The method for extracting neoantigens for immunotherapy according to claim 1, wherein the step S2 of acquiring nucleotide polymer sequence libraries of the tumor tissue sample and the normal tissue sample and a specific proteome of the tumor tissue sample comprises:
step S21: generating nucleotide polymer sequence libraries of a preset length;
step S22: acquiring tumor-specific nucleotide polymer sequences;
step S23: assembling the tumor-specific nucleotide polymer sequences to obtain assembled tumor-specific nucleotide polymer sequences; and
step S24: conducting reading frame translation on the assembled tumor-specific nucleotide polymer sequences.

4. The method for extracting neoantigens for immunotherapy according to claim 1, wherein the step S3 of acquiring the plurality of candidate tumor-specific neoantigens based on the conventional proteome of the tumor tissue sample and the specific proteome of the tumor tissue sample, and acquiring molecular HLA typing comprises:
step S31: acquiring the molecular HLA typing;
step S32: generating a global tumor proteome based on the conventional proteome of the tumor tissue sample and the specific proteome of the tumor tissue sample;
step S33: predicting HLA-peptide binding affinity scores to acquire a target peptide sequence using the global tumor proteome and the molecular HLA typing acquired; and
step S34: annotating characteristics of the target peptide sequence to acquire the plurality of candidate tumor-specific neoantigens.

5. A system for extracting neoantigens for immunotherapy, comprising:
a conventional proteome acquiring unit, used for acquiring a conventional proteome of a tumor tissue sample and a conventional proteome of a normal tissue sample;
a specific proteome acquiring unit, used for acquiring nucleotide polymer sequence libraries of the tumor tissue sample and the normal tissue sample, and a specific proteome of the tumor tissue sample;
a candidate neoantigen determining unit, used for acquiring a plurality of candidate tumor-specific neoantigens based on the conventional proteome of the tumor tissue sample and the specific proteome of the tumor tissue sample, and for acquiring molecular human leukocyte antigen (HLA) typing; and
a tumor-specific neoantigen determining unit, used for separately calculating feature values of the plurality of candidate tumor-specific neoantigens based on the plurality of candidate tumor-specific neoantigens acquired, and acquisition of tumor-specific neoantigens by filtering under a preset rule.

6. The system for extracting neoantigens for immunotherapy according to claim 5, wherein the conventional proteome acquiring unit comprises:
a detection subunit, used for detecting point mutations of transcripts of the tumor tissue sample and the normal tissue sample;
a calculation subunit, used for calculating expression levels of the transcripts in the tumor tissue sample and the normal tissue sample;
a construction subunit, used for constructing mutated exomes of the tumor tissue sample and the normal tissue sample; and
a translation subunit, used for translating the mutated exomes of the tumor tissue sample and the normal tissue sample.

7. The system for extracting neoantigens for immunotherapy according to claim 5, wherein the specific proteome acquiring unit comprises:
a generation subunit, used for generating the nucleotide polymer sequence libraries of a preset length;
an acquisition subunit, used for acquiring tumor-specific nucleotide polymer sequences;
an assembly subunit, used for assembling the tumor-specific nucleotide polymer sequences; and
a reading frame translation subunit, used for reading frame translation of the assembled tumor-specific nucleotide polymer sequences.

8. The system for extracting neoantigens for immunotherapy according to claim 5, wherein the candidate neoantigen determining unit comprises:
an HLA acquiring subunit, used for acquiring the molecular HLA typing;
a global tumor proteome generating subunit, used for generating a global tumor proteome based on the conventional proteome of the tumor tissue sample and the specific proteome of the tumor tissue sample;
a target peptide sequence acquiring subunit, used for predicting HLA-peptide binding affinity scores to acquire a target peptide sequence using the global tumor proteome and the molecular HLA typing acquired; and
a candidate tumor-specific neoantigen acquiring subunit, used for annotating characteristics of the target peptide sequence to acquire the plurality of candidate tumor-specific neoantigens.
